(19) United States
(12) Patent Application Publication
(10) Pub. No.: US 2013/0023716 A1
(43) Pub. Date: Jan. 24, 2013

(54) DOSE CONTROL SYSTEM AND METHOD OF CONTROLLING OUTPUT DOSE OF A RADIATION THERAPY TREATMENT SYSTEM

(71) Applicants: Mark Thomas, Madison, WI (US); James Zhang, Oconomowoc, WI (US); Megan Buroker, Gurnee, IL (US); Thomas Mathew, Johnson Creek, WI (US); Graham Lukey, Middleton, WI (US)

(72) Inventors: Mark Thomas, Madison, WI (US); James Zhang, Oconomowoc, WI (US); Megan Buroker, Gurnee, IL (US); Thomas Mathew, Johnson Creek, WI (US); Graham Lukey, Middleton, WI (US)

(21) Appl. No.: 13/456,052

(22) Filed: Apr. 25, 2012

Related U.S. Application Data
(60) Provisional application No. 61/478,839, filed on Apr. 25, 2011.

(51) Int. Cl. A61N 5/10

(52) U.S. Cl. 600/1

(57) ABSTRACT

A dose control system for a radiation therapy treatment system. The dose control system monitors the dose output and addresses the effects of rotational dose variation and drift.

### CROSS-REFERENCE TO RELATED APPLICATIONS

This application claims the benefit of, and priority to, U.S. Provisional Patent Application No. 61/478,839, filed on Apr. 25, 2011, the entire contents of which are incorporated herein by reference.

### FIELD OF THE INVENTION

The invention generally relates to radiation delivery and treatment systems, and more particularly to a dose control system and method of controlling an output dose of a helical radiation delivery system.

### BACKGROUND OF THE INVENTION

Helical radiation delivery systems have an advantage in delivering a precisely-controlled dose of radiation. One disadvantage of helical radiation delivery systems is the complexity of accounting for drift and rotational dose variation that is characteristic of radiation therapy in a helical delivery system.

### SUMMARY OF THE INVENTION

The radiation therapy treatment system described herein is a helical radiation delivery system that provides for time-based delivery of x-ray output. Currently, helical systems operate without closed-loop control of the dose output (i.e., the amount of dose exiting a linear accelerator). Neither the RF (radio frequency) input power to the linear accelerator (LINAC) nor the injected electron beam current is directly controlled by their respective power systems. The conventional helical system simply monitors the dose output to verify that it is within an allowable range.

The present invention relates to a dose control system that monitors the dose output and addresses the effects of rotational dose variation and drift. Drift (i.e., generally downwards) is caused by a temperature change within the electron gun that injects electrons into the acceleration field of the LINAC. Rotational dose variation, which is an oscillation (i.e., that looks like a sine wave) that corresponds to the gantry period, occurs due to the rotation of the LINAC. Gravity acts on the magnetron which causes fluctuations in the RF power to the LINAC.

In one embodiment, the present invention provides a dose control system for a radiation therapy system. The dose control system includes a sensor, a dose monitoring device, and a computer in communication with the sensor and the dose monitoring device. The computer includes a processor and a software program stored on a non-transitory computer readable medium accessible by the processor. The software program is operable to receive, from the dose monitoring device, a first input related to a measured dose value output from a linear accelerator of the radiation therapy system, and receive, from the sensor, a second input related to a measured electrical current value output from an electron gun of the radiation therapy system to the linear accelerator. The program is further operable to process the measured dose value output from the linear accelerator and the measured electrical current value output from the electron gun, determine a first output for controlling an amount of RF power applied to the linear accelerator based on the measured dose value output from the linear accelerator, and determine a second output for controlling an amount of electrical current applied to the electron gun based on the measured electrical current value output from the electron gun.

In another embodiment, the present invention provides a radiation therapy treatment system. Thy system includes a gantry, a radiation delivery device supported by the gantry, a couch configured to support a patient, a computer in communication with the gantry, the radiation delivery device, and the couch. The computer includes a processor and a software program stored on a non-transitory computer readable medium accessible by the processor. The software program is operable to determine a first input related to a measured dose value output from a linear accelerator of the radiation therapy system, and determine a second input related to a measured electrical current value output from an electron gun of the radiation therapy system to the linear accelerator. The program is further operable to process the measured dose value output from the linear accelerator and the measured electrical current value output from the electron gun, determine a first output for controlling an amount of RF power applied to the linear accelerator based on the measured dose value output from the linear accelerator, and determine a second output for controlling an amount of electrical current applied to the electron gun based on the measured electrical current value output from the electron gun.

In yet another embodiment, the present invention provides a method of controlling dose output to a patient receiving radiation therapy delivered by a radiation therapy system. The method includes measuring a dose value output from a linear accelerator of the radiation therapy system, determining an electrical current value output from an electron gun of the radiation therapy system to the linear accelerator, processing the measured dose value output from the linear accelerator and the electrical current value output from the electron gun. The method also includes generating a first transfer function to relate the measured dose value output from the linear accelerator to a radio frequency power value input to the linear accelerator, and generating a second transfer function to relate the electrical current value output from the electron gun to an electrical current value input to the electron gun. The method further includes determining a first correction value, based on the first transfer function, to apply to the radio frequency power value, determining a second correction value, based on the second transfer function, to apply to the electrical current value input to the electron gun, and applying the first correction value and the second correction value.

Other aspects of the invention will become apparent by consideration of the detailed description and accompanying drawings.

### BRIEF DESCRIPTION OF THE DRAWINGS

FIG. 1 is a graphical illustration comparing output dose of a radiation therapy system utilizing a dose control system and output dose of a radiation therapy system not utilizing the dose control system.

FIG. 2 is a perspective view of a radiation therapy treatment system.

FIG. 2A is a schematic diagram of the radiation therapy treatment system.

FIG. 3 is a block diagram illustrating the operation of a dose control system utilizing electron gun current feedback and measured output dose feedback according to one embodiment of the present invention.

FIG. 4 is a block diagram of the radiation therapy system of FIGS. 2 and 2A with the dose control system of FIG. 3.

FIG. 5 is a block diagram of operation of the dose control system utilizing electron gun current feedback and measured output dose feedback according to one embodiment of the present invention.

FIG. 6 is a graphical illustration showing how a transfer function used in the dose control system is derived.

FIG. 7 is a graphical illustration depicting why a radiation therapy system does not compensate its dose output for a dropped pulse.

FIG. 8 is a flow chart of a method of controlling dose output to a patient delivered by the radiation therapy system according to an embodiment of the invention.

### DETAILED DESCRIPTION OF THE INVENTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted,” “connected,” “supported,” and “coupled” and variations thereof are used broadly and encompass both direct and indirect mountings, connections, supports, and couplings.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form. In addition, terms such as “first,” “second,” and “third” are used herein for purposes of description and are not intended to indicate or imply relative importance or significance.

In addition, it should be understood that embodiments of the invention may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic based aspects of the invention may be implemented in software (e.g., stored on non-transitory computer-readable medium). As such, it should be noted that a plurality of hardware and software based devices, as well as a plurality of different structural components may be utilized to implement the invention. Furthermore, and as described in subsequent paragraphs, the specific mechanical configurations illustrated in the drawings are intended to exemplify embodiments of the invention and that other alternative mechanical configurations are possible.

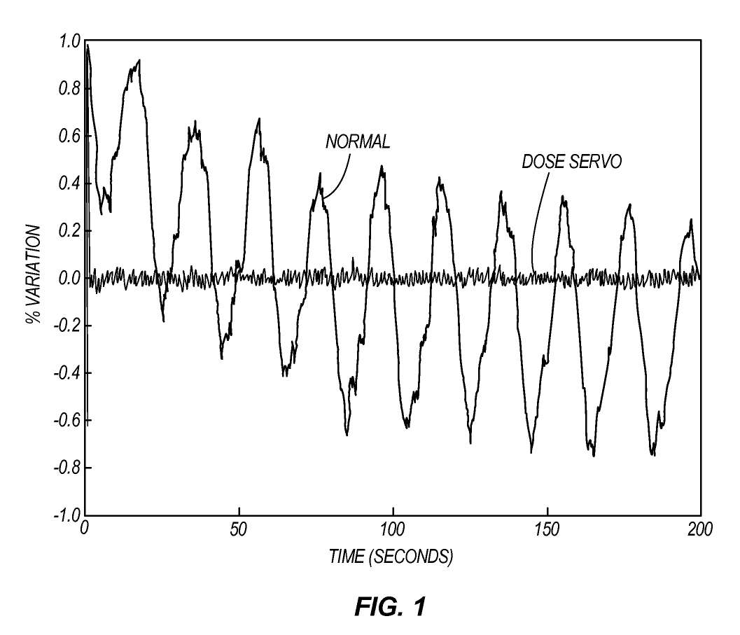

FIG. 1 illustrates the difference in rotational output variation and drift of a helical radiation delivery system with and without a dose control system. Note that without a dose control system (the line indicated as “Normal”), the helical system demonstrates a dose output having roughly ±0.8% rotational variation along with drift (generally downwards). During a treatment procedure on a patient, implementation of the proposed dose control system (the line indicated as “Dose Servo”) causes this rotational variation to drop to less than ±0.1% and the drift is almost completely removed.

The proposed dose control system of the present invention provides a significant reduction in rotational dose variation and drift in the helical radiation therapy treatment system. In addition, the dose control system can reduce dose variation for static procedures. One purpose of the dose control system is to stabilize output dose to ±0.5% by implementing a closed loop dose control system to adjust for the thermal and mechanical effects that impart variability on the x-ray output.

As explained in more detail below, the dose control system provides continuous, automatic control of the LINAC output through a closed-loop, servo control system. This servo control system relies upon dose monitoring feedback from the linear accelerator to control the input signals of the linear accelerator, providing consistent, stable output.

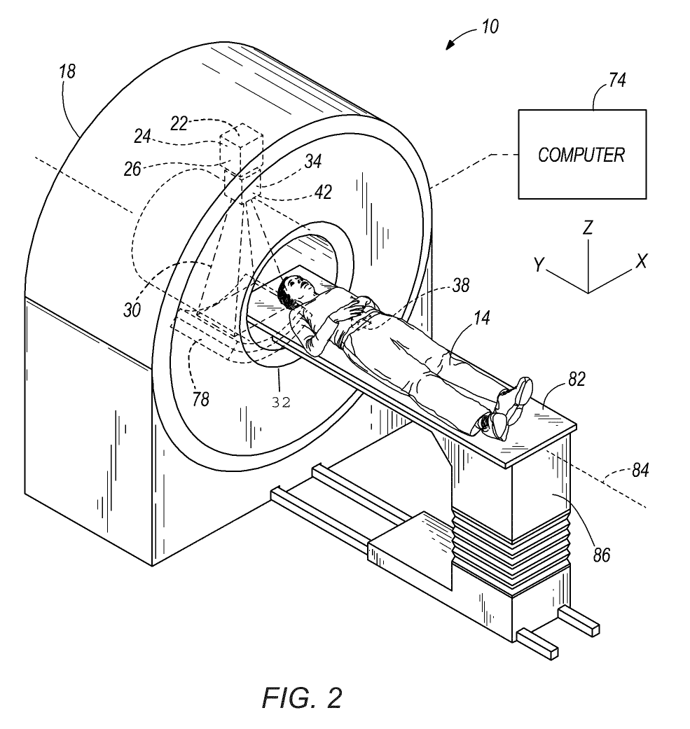

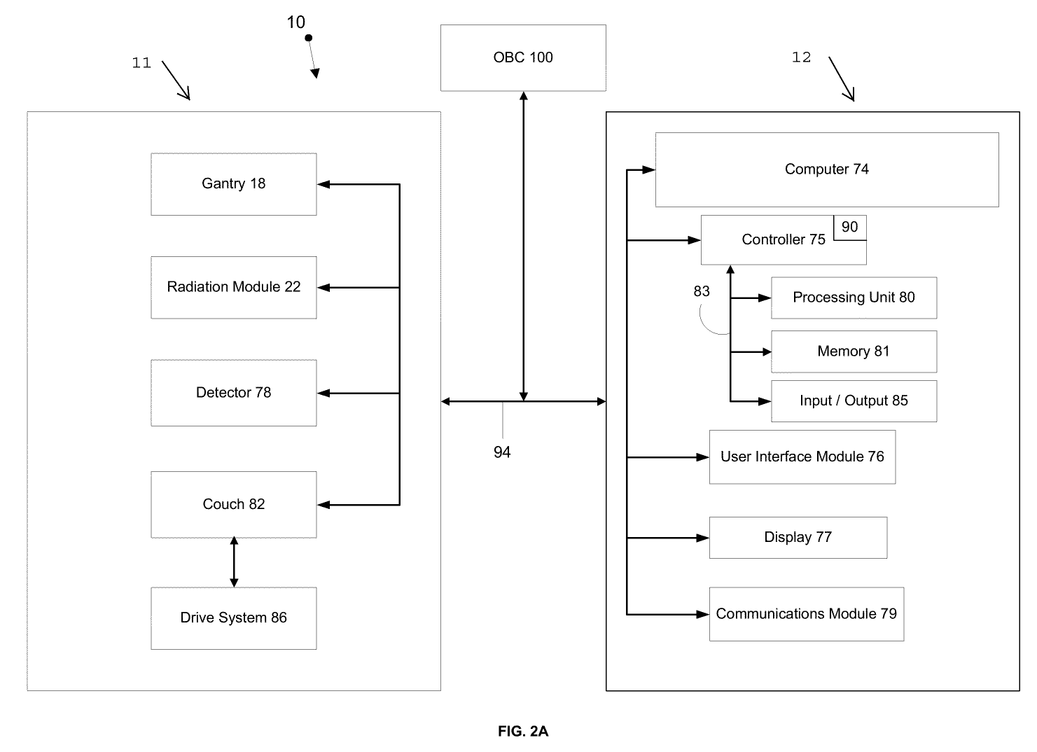

FIGS. 2 and 2A illustrate a helical radiation therapy treatment system 10 that provides radiation therapy to a patient 14 according to one embodiment of the present invention. The radiation therapy treatment system 10 includes a treatment delivery system 11 and a treatment planning system 12. The radiation therapy treatment (e.g., megavoltage energies) can include photon-based radiation therapy, brachytherapy, electron beam therapy, proton, neutron, particle therapy, or other types of treatment therapy.

The radiation therapy treatment system 10 includes a gantry 18. The gantry 18 supports a radiation module 22, which includes a radiation source 24 and a LINAC 26 that generates a beam 30 of radiation (e.g., megavoltage energies or kilovoltage energies). Although the gantry 18 shown in FIG. 2 is a ring gantry (i.e., it extends through a full 360° arc to create a complete ring or circle), other types of mounting arrangements may also be employed. For example, a C-type, partial ring gantry, or robotic arm gantry arrangement could be used. Any other framework capable of positioning the radiation module 22 at various rotational and/or axial positions relative to the patient 14 may also be employed. In addition, the radiation source 24 may travel in a path that does not follow the shape of the gantry 18. For example, the radiation source 24 may travel in a non-circular path even though the illustrated gantry 18 is generally circular-shaped. The gantry 18 of the illustrated embodiment defines a gantry aperture 32 into which the patient 14 moves during treatment.

The radiation module 22 also includes a modulation device 34 operable to modify or modulate the radiation beam 30. The modulation device 34 modulates the radiation beam 30 and directs the radiation beam 30 toward the patient 14. Specifically, the radiation beam 30 is directed toward a portion 38 of the patient 14. The portion 38 may include the patient's entire body, but is generally smaller than the patient's entire body and can be defined by a two-dimensional area and/or a three-dimensional volume. A portion may include one or more regions of interest. For example, a region desired to receive the radiation, which may be referred to as a target or target region, is an example of a region of interest. Another type of region of interest is a region at risk. If a portion includes a region at risk, the radiation beam is preferably diverted from the region at risk. The patient 14 may also have more than one target region that needs to receive radiation therapy. Such modulation is sometimes referred to as intensity modulated radiation therapy (“IMRT”).

The portion 38 may include or be referred to as a target or target region or a region of risk. If the portion 38 includes a region at risk, the radiation beam 30 is preferably diverted from the region at risk. Such modulation is sometimes referred to as intensity modulated radiation therapy (“IMRT”).

The radiation therapy treatment system 10 can also include a detector 78 (e.g., a kilovoltage or a megavoltage detector), as illustrated in FIG. 2, that receives the radiation beam 30. The linear accelerator 26 and the detector 78 can also operate as a computed tomography (“CT”) system to generate CT images of the patient 14. The linear accelerator 26 emits the radiation beam 30 toward the portion 38 of the patient 14. The portion 38 absorbs some of the radiation. The detector 78 detects or measures the amount of radiation absorbed by the portion 38. The detector 78 collects the absorption data from different angles as the linear accelerator 26 rotates around and emits radiation toward the patient 14. The collected absorption data is transmitted to a computer 74, and the computer 74 processes the collected adsorption data to generate images of the patient's body tissues and organs. The images can also illustrate bone, soft tissues, and blood vessels.

The CT images can be acquired with a radiation beam 30 that has a fan-shaped geometry, a multi-slice geometry or a cone-beam geometry. In addition, the CT images can be acquired with the linear accelerator 26 delivering megavoltage energies or kilovoltage energies. It is also noted that the acquired CT images can be registered with previously acquired CT images (from the radiation therapy treatment system 10 or other image acquisition devices, such as other CT scanners, MRI systems, and PET systems). For example, the previously acquired CT images for the patient 14 can include identified targets 38 made through a contouring process. The newly acquired CT images for the patient 14 can be registered with the previously acquired CT images to assist in identifying the targets 38 in the new CT images. The registration process can use rigid or deformable registration tools.

The image data can be presented on a display as either a three-dimensional image or a series of two-dimensional images. In addition, the image data comprising the images can be either voxels (for three-dimensional images) or pixels (for two-dimensional images). The term image element is used generally in the description to refer to both.

The system 10 can also include a patient support device, shown as a couch 82 in FIG. 2, to support at least a part of the patient 14 during treatment. For example, while the illustrated couch 82 is designed to support the patient's entire body, in other embodiments of the invention, the patient support device can be designed to support only a part of the patient 14 during treatment. The couch 82, or at least portions thereof, moves into and out of the field of radiation along an axis 84 (along the Y axis). The couch 82 is also capable of moving along the X and Z axes as illustrated in FIG. 2. The radiation delivery system 10 also can include a drive system 86 operable to manipulate the position of the couch 82. The drive system 86 can be controlled by the computer 74.

As shown in FIGS. 2 and 2A, the radiation therapy system 10 includes the computer 74, which is embodied as an operator station to be accessed by medical personnel. With continued reference to FIG. 2A, the computer 74 includes a controller 75, a user interface module 76, a display 77, and a communications module 79. The controller 75 and the user interface module 76 include combinations of software and hardware that are operable to, among other things, control the operation of the radiation delivery system 10 and the information that is presented on the display 77.

The controller 75 includes, for example, a processing unit 80 (e.g., a microprocessor, a microcontroller, or another suitable programmable device), a memory 81, and a bus 83. The bus 83 connects various components of the controller 75, including the memory 81, to the processing unit 80. The processing unit 80 may represent one or more general-purpose processors, a special purpose processor such as a digital signal processor or other type of device such as a controller or field programmable gate array.

It should be understood that although the controller 75, the user interface module 76, the display 77, and the communications module 79 are illustrated as part of a single server or computing device, the components of the radiation delivery system 10 can be distributed over multiple servers or computing devices. Similarly, the radiation delivery system 10 can include multiple controllers 75, user interface modules 76, displays 77, and communications modules 79.

The memory 81 includes, for example, a read-only memory (“ROM”), a random access memory (“RAM”), an electrically erasable programmable read-only memory (“EEPROM”), a flash memory, a hard disk, an SD card, or another suitable magnetic, optical, physical, or electronic memory device. The processing unit 80 is connected to the memory 81 and executes software program 90 that is capable of being stored in the RAM (e.g., during execution), the ROM (e.g., on a generally permanent basis), or another non-transitory computer readable medium such as another memory or a disc. Additionally or alternatively, the memory 81 is included in the processing unit 80. The controller 75 also includes an input/output (“I/O”) system 85 that includes routines for transferring information between components within the controller 75 and other components of the system 10. Software included in the implementation of the system 10 is stored in the memory 81 of the controller 75. The software includes, for example, firmware, one or more applications, program data, one or more program modules, and other executable instructions. The controller 75 is configured to retrieve from memory and execute, among other things, instructions related to the methods described below.

The user interface module 76 is configured for user control of the system 10 and to input various parameters into the radiation treatment system 10. For example, the user interface module 76 is operably coupled to the controller 75 to control the information presented on the display 77. The user interface module 76 can include a combination of digital and analog input or output devices required to achieve a desired level of control for the radiation treatment system 10. For example, the user interface module 76 can include input devices such as a touch-screen display, a plurality of knobs, a plurality of dials, a plurality of switches, a plurality of buttons, or the like.

The display 77 is, for example, a liquid crystal display (“LCD”), a light-emitting diode (“LED”) display, an organic LED (“OLED”) display, an electroluminescent display (“ELD”), a surface-conduction electron-emitter display (“SED”), a field emission display (“FED”), a thin-film transistor (“TFT”) LCD, or the like. In other constructions, the display 77 is a Super active-matrix OLED (“AMOLED”) display.

In some implementations, the radiation treatment system 10 and the computer 74 are also configured to connect to a network 94 (e.g., a WAN, a LAN, or the like) via the communications module 79 to access other programs, software, computers, or other radiation treatment systems 10. For example, the computer 74 can communicate with an on-board computer (OBC) 100, which controls certain functionalities of the dose control system 105. As explained in more details below, the OBC 100 includes a software program(s) 104 that includes instructions for performing closed-loop control of injected gun current and dose output to account for drift and rotational variation.

The communications module 79 can include a network interface, such as an Ethernet card or a wireless network card, that allows the radiation treatment system 10 to send and receive information over a network, such as a local area network or the Internet. In some embodiments, the communications module 79 includes drivers configured to receive and send data to and from various input and/or output devices, such as a keyboard, a mouse, a printer, etc. Data communications can occur via a wireless local area network (“LAN”) using any of a variety of communications protocols, such as Wi-Fi, Bluetooth, ZigBee, or the like. Additionally or alternatively, data communications can occur over a wide area network (“WAN”) (e.g., a TCP/IP based network or the like).

The communications module 79 is also compatible with the Digital Imaging and Communications in Medicine (DICOM) protocol with any version and/or other required protocol. DICOM is an international communications standard developed by NEMA that defines the format used to transfer medical image-related data between different pieces of medical equipment. DICOM RT refers to the standards that are specific to radiation therapy data.

The two-way arrows in FIG. 2A generally represent two-way communication and information transfer where indicated. However, for some medical and computerized equipment, only one-way communication and information transfer may be necessary.

The processing unit 80 executes instructions stored in the computer-readable media. The instructions can include various components or modules configured to perform particular functionality when executed by the processing unit 80. For example, the software program 90 includes a plurality of modules or applications that interact or communicate with one another to provide instructions to the processing unit 80 for generating a treatment plan for a patient, modifying or adapting a treatment plan, acquiring images of the patient, and controlling the components of the radiation system 10 to delivering radiation to the patient and to monitoring the radiation dose output of the system.

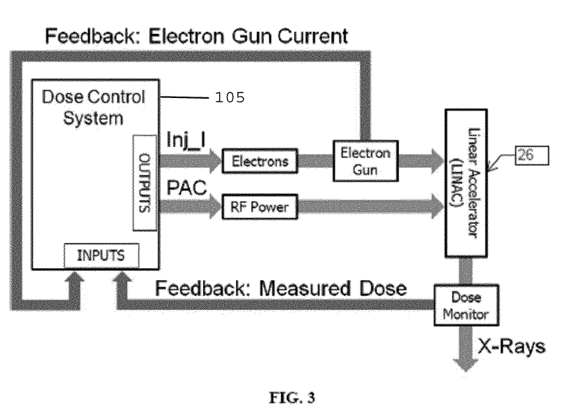

The proposed dose control system 105 as illustrated in FIGS. 3, 4, and 5 provides continuous, automatic control of the LINAC output dose. This control is achieved by establishing a “closed loop” control system, or servo. FIG. 3 illustrates a simplified block diagram of the relationship between the components in the dose control system 105. As shown in FIG. 3, in the servo, the dose control system 105 controls and adjusts two normally constant setup parameters that impact dose output. First, dose control system 105 controls and adjusts the Injected Current (Inj_I), input current to the electron gun—this signal, denoted Inj_I by the Solid State Modulator controller, controls the amount of electrons injected into the acceleration field of the linear accelerator. The Inj_I signal has the most prominent effect on the output energy. Second, dose control system 105 controls and adjusts the PAC (pulse amplitude control), the magnitude of the magnetron pulse—this signal, denoted PAC by the Solid State Modulator controller, excites (i.e., enables or regulates) the magnetron to create the RF power that establishes the acceleration field of the linear accelerator. The PAC signal has the most prominent effect on output dose. Using both the Inj_I and PAC signals, the dose control system 105 can react to feedback signals from the monitored dose and electron gun current signals to effectively control output dose and energy.

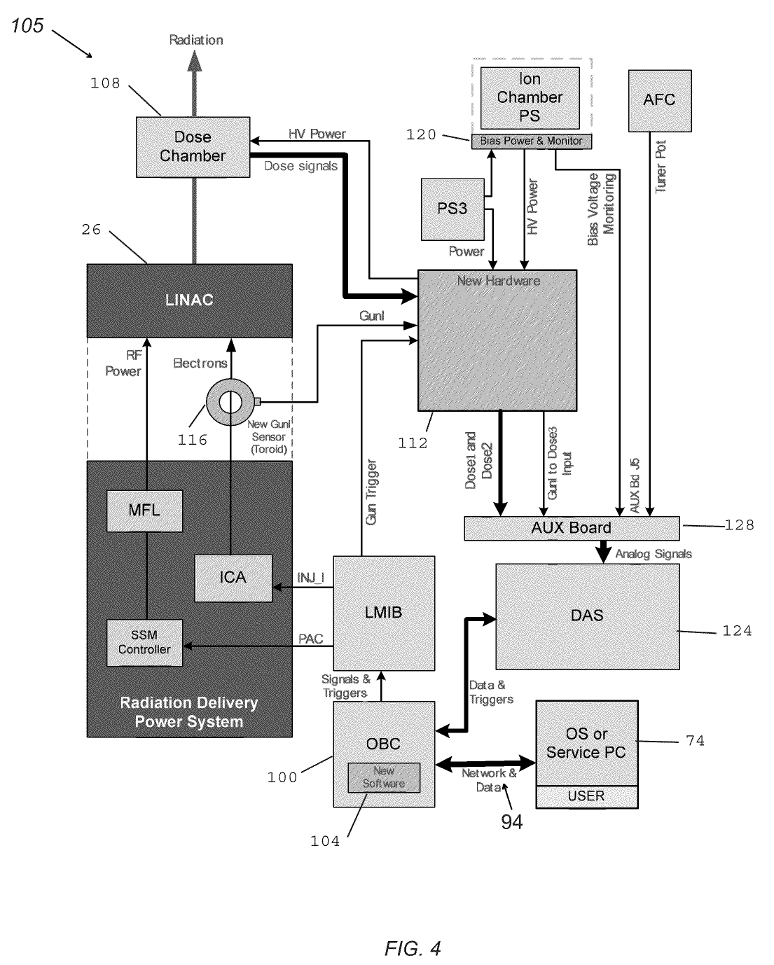

With reference to FIG. 4, the dose control system 105 is schematically illustrated in connection with some of the components of the radiation delivery system 10. As shown in FIG. 4, a RF input power and a beam with electrons is supplied to the LINAC 26 that generates a beam of radiation for treating the patient. The dose output of the system 10 is the radiation dose exiting the LINAC 26 and measured by the ion dose chamber 108. The dose control system 105 comprises, among other elements, an electronics assembly 112, a toroid sensor 116, and a power supply 120 for biasing the dose chamber 108. The new hardware of the dose control system 105 includes printed circuit boards, assemblies, and cables that create the closed-loop feedback system and support various interlocks (described below).

The toroid sensor 116 measures the current from the electron gun. The gun current signal measured by the toroid sensor 116 is brought back to a newly designed printed circuit assembly (PCA), which samples the signal and sends it to the data acquisition system (DAS) 124 (via the Aux Board 128) using the existing, but unused, Dose3 signal. The Aux Board 128 is a connection point for signals and auxiliary data. The PCA performs a sample and hold signal conversion. The PCA is housed in the electronics assembly 112 of the dose control system 105. The electronics assembly 112 also replaces the existing phenolic, or dose isolation, assembly. The phenolic assembly has traditionally provided a connection point for the dose ion chamber signals and high voltage bias.

To account for the dose ion chamber connections, there are three PCAs inside electronics assembly 112 of the dose control system 105. The first PCA is the PCA mentioned above. The second PCA allows the Dose1 and Dose2 signals from the dose ion chamber to be passed through to the DAS 124 (via the Aux Board 128) as well as terminates the four Dose3 signals from the dose ion chamber to 50Ω. The third PCA provides a pass through for the high voltage coming from the power supply 120 and going to the dose ion chamber 108.

The dose control system 105 also includes a modification to the existing power supply 120. The power supply 120 provides power to the elements of the dose control system 105. In one embodiment, the existing 500V power supply is replaced by a 1 kV supply 120 so that the dose ion chamber 108 can be biased at 600V. The power supply 120 is enclosed so that there are no longer any exposed pins carrying high voltage. This design also takes advantage of the monitoring capability of the power supply so that the OBC 100 can monitor the supply's output voltage and can interlock if the voltage is out of range. The bias voltage monitoring signal is brought back through J5 at the Aux Board 128.

An interlock cancels a procedure when a condition presents itself deemed to be out of range or tolerance or not representing expected behavior. The dose control system 105 adds four new interlocks:

1. Ion chamber bias voltage interlock: Using a monitor signal, the OBC 100 interlocks if the voltage supplied to the dose ion chamber 108 is outside of this range: 600V±10%.

2. Electron gun current interlock: The OBC 100 interlocks if the measured gun current is outside of its range (Nominal±5%.).

3. Consecutive number of dropped pulses: The OBC 100 interlocks if it detects two consecutive seconds of dropped pulses, where a pulse is considered dropped if it is 90% or lower than the nominal value.

4. Control parameter range limit: Boundaries have been set on how low or high Inj_I and PAC can be adjusted. If the servo has been using the maximum or minimum value for 5 seconds to adjust for the error, the OBC 100 will open the interlock.

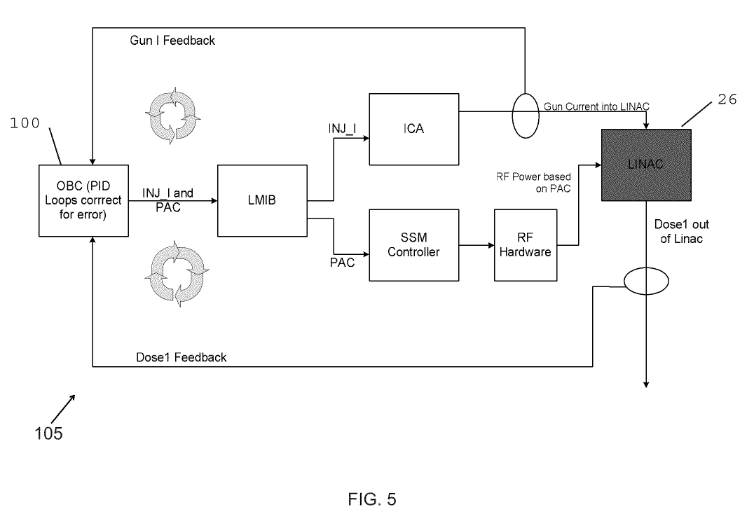

As schematically illustrated in FIGS. 4 and 5, the OBC 100 includes a software program(s) 104 that includes instructions for performing closed-loop control of injected gun current and dose output to account for drift and rotational variation. The software program 104 includes a PID (proportional, integral, derivative) control loop algorithm. In one embodiment, the OBC 100 uses feedback from the gun current being injected into the LINAC and Dose1 from the dose monitor ion chamber 108 to adjust Inj_I and PAC programming voltages, respectively, which relate to the measured outputs via a transfer function. In particular, the OBC 100 receives, from the dose monitor in the dose chamber 108, a first input related to the measured dose value output from the linear accelerator. Further, the OBC 100 receives from the sensor 116, a second input related to the measured electrical current value output from the electron gun of the radiation therapy system to the linear accelerator. The OBC 100 processes the measured dose value output from and the measured electrical current value output by using transferred functions as described below. The OBC 100 determines a first output (i.e., a first correction value) for controlling an amount of RF power applied to the linear accelerator based on the measured dose value output from the linear accelerator. Further, the OBC 100 determines a second output (i.e., a second correction value) for controlling an amount of electrical current applied to the electron gun based on the measured electrical current value output from the electron gun. Thus, the OBC 100 regulates the values of Inj_I and PAC control signals in order to control the dose output of the radiation delivery system 10.

The PID control loop algorithm is applied at both Image and Treatment level accelerator output modes (AOM). An Image level AOM is a set of machine parameters set up by the user to image a patient (e.g., like a CT machine does), using a lower level of dose, to see where the tumor is so that the patient can be correctly positioned. A treatment level AOM is a second set of machine parameters set up by the user to treat (i.e., actually radiate the tumor) the patient, using a higher level dose output.

The PID control loop algorithm uses transfer functions that relate Dose1 to PAC and Gun Current to Injector Current. These transfer functions are derived by a sampling, averaging and fitting technique. With the control loop disabled, the input variable is stepped through five consecutive levels that are 1% of nominal apart. The output variable is sampled at each level for five seconds, after which the average of the samples is calculated. The five input level/output average pairs are then used to calculate the slope and intercept of a line using a least-squares fit. The slope and intercept form the transfer function.

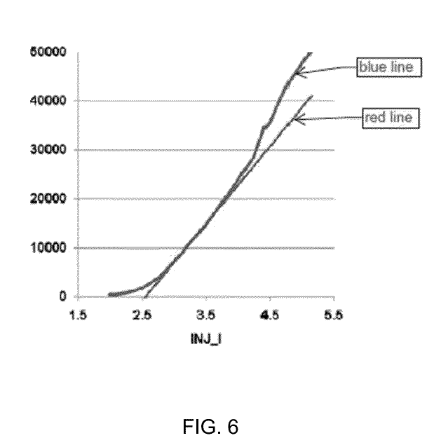

More specifically, a transfer function is obtained by measuring five points about a curve and fitting a line to it (see FIG. 6). The blue trace in FIG. 6 represents the Gun Current (“GunC”) vs. Inj_I on the radiation therapy system 10. The red trace in FIG. 6 represents the result of the procedure to obtain the transfer function—it is a linear approximation of the blue curve over a smaller operating range. An initial Inj_I is selected that is near where the radiation therapy system is run. For example, in FIG. 6, it might be around 3.5. The GunC value is then measured. The Inj_I is increased 1% and 2% and decreased 1% and 2% to get five points in the format (Inj_I, GunC) on the line. Using a least squares fit technique, the equation for a line is created (y=m×+b) where m is the slope and b is the y intercept. The slope (kₛ) is used in the equation of the PID control algorithm shown below.

The PID control loop algorithm includes four transfer functions as there are a different slope and intercept for Imaging Dose1/PAC, Treatment Dose1/PAC, Imaging GunC/Inj_I and Treatment GunC/Inj_I. These functions are obtained so that the PID loop (the actual control algorithm) knows how much of a change is should apply based on its calculated error.

The PID control loop algorithm equation looks like:

1/kₛ(kₚ*Error+kᵢ*Σₑᵣᵣ+k_{d}*Δₑᵣᵣ)=Correction

where:

kₛ is the slope of the transfer function,

kₚ is the constant used to multiply by the proportional error,

kᵢ is the constant used to multiply by the integral of the error, and

k_{d} is the constant used to multiply the derivative of the error.

These constants are different for the Dose1/PAC loop and the GunC/Inj_I loop.

Control range limits are applied to the PAC and Inj_I to ensure that the changes made to these parameters are not too large. The control range limits for a control loop are defined as the transfer-function-scaled output values that correspond to ±10% of the nominal value of the input variable. Procedures are not allowed to continue if the output remains at the control range limits for greater than five seconds.

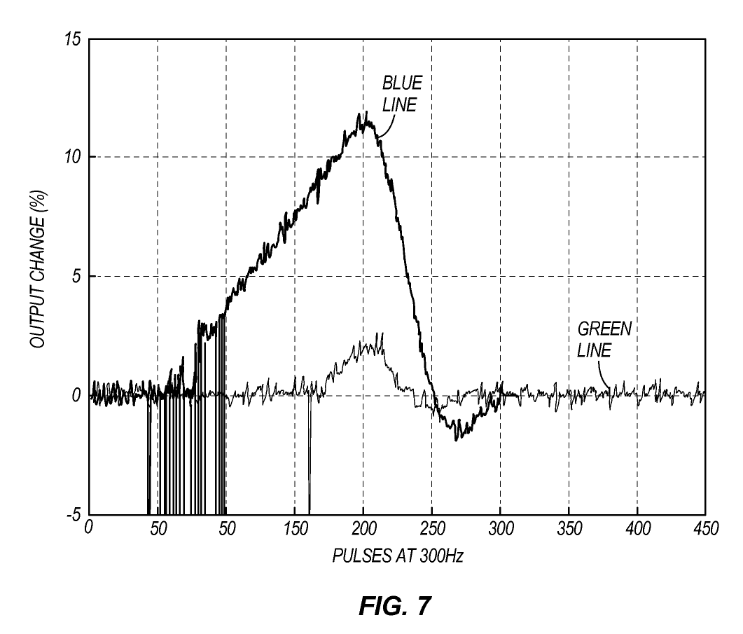

Finally, as the radiation dose is delivered (according to a treatment plan), some pulses throughout the treatment are dropped or missing. Instead of allowing this dropped pulse (an individual pulse that is below 90% of the nominal dose output) to skew the PID loop average of the buffer and create an inaccurate correction, the algorithm will ignore any value of Dose1 or Gun Current below 90% of their nominal value. The control loops insert the nominal value of Dose1 or Gun Current into the average buffer so that no change is made because of the dropped pulse. This is done because the dose control system 105 has a finite response time limited by the fixed pulse rate and the RF Power System capabilities; thus, the correction would be delivered at an incorrect gantry position. This basically means that the radiation therapy system 10 does not have the ability to compensate for a dropped pulse fast enough. If the dropped pulses (the lines shooting downwards in FIG. 7) are not ignored, the dose missing from the dropped pulses is compensated for and ends up an incorrect dose output being delivered at the wrong gantry position (again illustrated by the blue trace in FIG. 7). The green trace in FIG. 7 reflects that even with one dropped pulse (the line shooting downwards), the PID loop gives more dose in the next 100 pulses (over a ˜⅓ of a second) to accommodate for the dose that was missed. This illustrates why the algorithm now ignores dropped pulses and so that there is no overdose or dose delivered in the wrong gantry position.

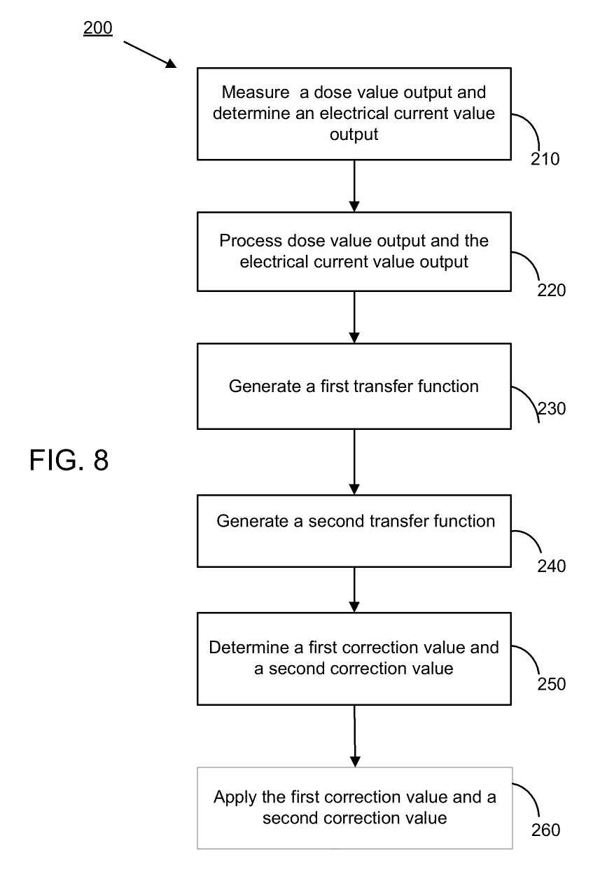

FIG. 8 is a flow chart illustrating a method 200 of controlling dose output to a patient receiving radiation therapy delivered by the radiation therapy system 10. The method 200 can be executed by a processor of the OBC 100 or the processing unit 80 of the controller 75 or a combination of both. Various steps described herein with respect to the method 200 are capable of being executed simultaneously, in parallel, or in an order that differs from the illustrated serial manner of execution. The method 200 is also capable of being executed using additional or fewer steps than are shown in the illustrated embodiment.

The first step in the method 200 of controlling dose output is measuring a dose value output from a linear accelerator 26 and determining an electrical current value output from the electron gun to the linear accelerator 26 (at step 210). In some embodiments, the dose value output is measured by the dose monitor of the dose chamber 108 and the electrical current value output is determined by the toroid sensor 116. Next, the processor 80 processes the measured dose value output from the linear accelerator and the electrical current value output from the electron gun (at step 220). In one embodiment, the processor uses the steps described above and generates a first transfer function to relate the measured dose value output from the linear accelerator to a radio frequency power value input to the linear accelerator (at step 230). Then, the processor generates a second transfer function to relate the electrical current value output from the electron gun to an electrical current value input to the electron gun (at step 240).

In the next step, the processor determines a first correction value, based on the first transfer function (at step 250). Next, the processor determines a second correction value, based on the second transfer function (at step 250). Then, the processor applies first correction value to the radio frequency power value and the second correction value to the electrical current value input to the electron gun. That way, the dose control system 105 controls the dose output delivered by the radiation therapy system 10.

Although the invention has been described in detail with reference to certain preferred embodiments, variations and modifications exist within the scope and spirit of one or more independent aspects of the invention as described.

What is claimed is:

1. A dose control system for a radiation therapy system, the dose control system comprising:
a sensor;
a dose monitoring device;
a computer in communication with the sensor and the dose monitoring device, the computer including a processor; and
a software program stored on a non-transitory computer readable medium accessible by the processor, the software program being operable to
receive, from the dose monitoring device, a first input related to a measured dose value output from a linear accelerator of the radiation therapy system,
receive, from the sensor, a second input related to a measured electrical current value output from an electron gun of the radiation therapy system to the linear accelerator,
process the measured dose value output from the linear accelerator and the measured electrical current value output from the electron gun,
determine a first output for controlling an amount of RF power applied to the linear accelerator based on the measured dose value output from the linear accelerator, and
determine a second output for controlling an amount of electrical current applied to the electron gun based on the measured electrical current value output from the electron gun.

2. The dose control system of claim 1, wherein the first output includes a pulse amplitude control signal.

3. The dose control system of claim 1, wherein the second output includes an injected current control signal.

4. The dose control system of claim 3, wherein the injected current control signal controls the amount of electrons injected into an acceleration field of the linear accelerator.

5. The dose control system of claim 1, wherein the sensor is a toroid sensor.

6. The dose control system of claim 1, wherein the dose monitoring devise is a dose monitor of a dose ion chamber receiving input from the linear accelerator.

7. The dose control system of claim 1, further comprising an electronics assembly including at least three printed circuit boards.

8. The dose control system of claim 1, further comprising an enclosed power supply that is at least 1 kV.

9. The dose control system of claim 1, wherein the processor uses at least one transfer function derived by a sampling, averaging, and fitting algorithm to determine the first and the second output.

10. The dose control system of claim 1, wherein the processor applies control range limits to the pulse amplitude control signal and the injected current control signal.

11. The dose control system of claim 1, wherein the processor ignores the first input and the second input when these values are below about ninety percent of their nominal value.

12. A radiation therapy treatment system comprising:
a gantry;
a radiation delivery device supported by the gantry;
a couch configured to support a patient;
a computer in communication with the gantry, the radiation delivery device, and the couch, the computer including a processor; and
a software program stored on a non-transitory computer readable medium accessible by the processor, the software program being operable to
determine a first input related to a measured dose value output from a linear accelerator of the radiation therapy system,
determine a second input related to a measured electrical current value output from an electron gun of the radiation therapy system to the linear accelerator,
process the measured dose value output from the linear accelerator and the measured electrical current value output from the electron gun,
determine a first output for controlling an amount of RF power applied to the linear accelerator based on the measured dose value output from the linear accelerator, and
determine a second output for controlling an amount of electrical current applied to the electron gun based on the measured electrical current value output from the electron gun.

13. The radiation therapy treatment system of claim 12, wherein the first output includes a pulse amplitude control signal.

14. The radiation therapy treatment system of claim 12, wherein the second output includes an injected current control signal.

15. The radiation therapy treatment system of claim 12, further comprising a toroid sensor that determines the first input.

16. The radiation therapy treatment system of claim 12, further comprising a dose monitor of configured to determine the second input.

17. The radiation therapy treatment system of claim 12, wherein the processor uses at least one transfer function derived by a sampling, averaging, and fitting algorithm to determine the first and the second output.

18. The radiation therapy treatment system of claim 12, wherein the processor applies control range limits to the pulse amplitude control signal and the injected current control signal.

19. The radiation therapy treatment system of claim 12, wherein the processor ignores the first input and the second input when these values are below ninety percent of their nominal value.

20. A method of controlling dose output to a patient receiving radiation therapy delivered by a radiation therapy system, the method comprising:
measuring a dose value output from a linear accelerator of the radiation therapy system;
determining an electrical current value output from an electron gun of the radiation therapy system to the linear accelerator;
processing the measured dose value output from the linear accelerator and the electrical current value output from the electron gun;
generating a first transfer function to relate the measured dose value output from the linear accelerator to a radio frequency power value input to the linear accelerator;
generating a second transfer function to relate the electrical current value output from the electron gun to an electrical current value input to the electron gun;
determining a first correction value, based on the first transfer function, to apply to the radio frequency power value;
determining a second correction value, based on the second transfer function, to apply to the electrical current value input to the electron gun; and
applying the first correction value and the second correction value.
